(19) United States
(12) Patent Application Publication
(10) Pub. No.: US 2013/0052175 A1
(43) Pub. Date: Feb. 28, 2013

(54) OIL SUSPENSION-BASED MOLDED FOOD PRODUCTS

(71) Applicant: Gil Ben Yosef, Even Yehuda (IL)

(72) Inventor: Gil Ben Yosef, Even Yehuda (IL)

(73) Assignees: MAABAROT PRODUCTS LTD., Kibbutz Maabarot (IL); ANLIT LTD., M. P. Hefer (IL); BIOPET LTD., Kibbutz Maabarot (IL)

(21) Appl. No.: 13/635,774

(22) PCT Filed: Jan. 17, 2011
(86) PCT No.: PCT/IL11/00051

§ 371 Date: Nov. 1, 2012

Related U.S. Application Data
(60) Provisional application No. 61/315,124, filed on Mar. 18, 2010.

(51) Int. Cl. A23L 1/30; A61K 36/02; A61K 31/7008; A61K 35/60; A61K 35/20; A61K 36/00; A61K 38/39; A61K 31/10; A61K 47/44; A61K 31/202; A61K 31/20; A23D 9/007; A23J 1/02; A23J 1/18; A23L 1/308; A23L 1/302; A23L 1/304; A23L 1/305; A23L 1/303; A23L 1/27; A23J 1/04; A23J 1/14; A23J 1/20; A23J 1/08; A61K 38/43

(52) U.S. Cl. 424/94.1; 424/195.17; 514/54; 514/62; 424/523; 424/535; 424/725; 514/17.2; 514/711; 514/773; 514/560; 514/558; 426/601; 426/656; 426/608; 426/657; 426/62; 426/648; 426/72; 426/61; 426/73; 426/250

(57) ABSTRACT

A molded food product is provided that has a high fat and protein content and a low carbohydrate content. The food product is suitable for human or animal consumption. The food product can serve as a snack or as a carrier for active ingredients such as vitamins, minerals, herbs and other food additives.

### CROSS-REFERENCE TO RELATED APPLICATIONS

Reference is made to U.S. Provisional Patent Application Ser. No. 61/315,124, filed Mar. 18, 2010 and entitled OIL SUSPENSION-BASED MOLDED FOOD PRODUCTS, the disclosure of which is hereby incorporated by reference and priority of which is hereby claimed pursuant to 37 CFR 1.78(a)(4) and (5)(i).

### FIELD OF THE INVENTION

The present invention relates to food products and food supplements. More specifically, the present invention relates to low carbohydrate oil suspension-based molded food products.

### BACKGROUND OF THE INVENTION

The following publications are believed to represent the current state of the art:
U.S. Pat. Nos. 4,199,611, 4,212,892, 4,256,690, and 5,501,865;
U.S. Patent Publication No. 2006/0051492 and 2009/0169695; and Published PCT Application: WO 04/107878.

### SUMMARY OF THE INVENTION

The present invention seeks to provide a molded food product for human or animal consumption that has a high protein content and a low carbohydrate content.

Accordingly, there is provided in accordance with one embodiment of the present invention a molded food product including 25-45% fat by weight, 30-70% protein by weight and not more than 20% carbohydrate by weight. Preferably, the water content of the product is less than 1% by weight, more preferably less than 0.5% by weight.

In accordance with a preferred embodiment of the present invention, the food product includes not more than 10% carbohydrate.

Preferably, the food product includes 30-35% fat. The fat is preferably selected from palm oil, cocoa butter, lard, coconut butter, beeswax, carnauba wax, tallow and combinations thereof. More preferably the fat is selected from palm oil, cocoa butter, coconut butter and combinations thereof.

In accordance with a preferred embodiment of the present invention, the food product includes 50-65% protein. Preferably, the protein is selected from poultry meal, poultry by-product meal, meat meal, fish meal, dehydrated whey protein concentrate, soy protein concentrate, dried yeast, brewer's yeast, egg powder, animal digest and combinations thereof. In one embodiment, the protein is selected from dehydrated whey protein concentrate, soy protein concentrate, brewer's yeast, egg powder and combinations thereof. In an alternative embodiment, the protein is selected from poultry meal, poultry by-product meal, fish meal, soy protein concentrate, brewer's yeast and egg powder.

Preferably, the weight percent in the product of the fat and the protein combined is from 60-98%, more preferably from 85-95%.

In accordance with a preferred embodiment of the present invention, the food product includes food additives selected from active ingredients, non-soluble fibers, food stabilizers, flavors and colors. Preferably, the product includes 2-38% by weight food additives, excluding the non-soluble fibers. More preferably, the product includes 2-20% by weight food additives, excluding the non-soluble fibers.

Preferably, the active ingredients are selected from minerals, vitamins, enzymes, amino acids, probiotic bacteria, fish oils, omega-3 fatty acids, fruits, algae extracts, vegetables, herbs and herb extracts, essential free fatty acids, joint active ingredients, colostrum and carotenoids. In one embodiment, the active ingredients are selected from vitamins, probiotic bacteria, joint active ingredients, fish oils, omega-3 and omega-6 fatty acids, herbs and herb extracts, colostrum and carotenoids. In an alternative embodiment, the active ingredients are selected from probiotic bacteria, oil soluble vitamins, joint active ingredients and omega-3 and omega-6 fatty acids.

In accordance with a preferred embodiment of the present invention, the non-soluble fibers are selected from fructo-oligosaccharides (FOS), gluco-oligosaccharides (GOS), long-chain oligosaccharide polymers of fructose, long-chain oligosaccharide polymers of glucose and raffinose. Preferably, the product includes not more than 10% non-soluble fibers.

Preferably, the food stabilizers include lecithin. Preferably, the flavors include palatability enhancers.

In accordance with a preferred embodiment of the present invention, the food product is in the form of an oil suspension. Preferably, the particles in the product have a maximum dimension of not more than 100 microns, more preferably not more than 50 microns. Preferably, the food product is a solid at temperatures below 40° C.

There is provided in accordance with a further embodiment of the present invention a packaged food product including: a tray having a plurality of cavities formed therein and a cover sheet, wherein the cavities contain the molded food product described hereinabove.

Preferably, the tray includes a material selected from polyvinyl chloride (PVC), polyethylene terephthalate (PET), a polyethylene and PVC laminate, a polyester and an aluminum and polyethylene laminate. Preferably, the cover sheet includes a material selected from laminated foil, polyethylene and polypropylene laminate or aluminum foil.

There is provided in accordance with an additional embodiment of the present invention a method of forming a molded food product including: melting a plant or animal fat, mixing the melted plant or animal fat with a protein source to form a premix, reducing the size of the particles in the premix to generate an oil suspension, and pouring the oil suspension into cavities of packages to form the molded food product.

Preferably, the plant or animal fat is selected from palm oil, cocoa butter, lard, coconut butter, beeswax, carnauba wax, tallow and combinations thereof Preferably, the protein source is selected from poultry meal, poultry by-product meal, meat meal, fish meal, dehydrated whey protein concentrate, soy protein concentrate, dried yeast, brewer's yeast, egg powder, animal digest and combinations thereof.

In accordance with a preferred embodiment of the present invention, the reducing is performed by wet grinding. Preferably, the size of the particles is reduced to not more than 100 microns, more preferably not more than 50 microns.

Preferably, the packages include a material selected from polyvinyl chloride (PVC), polyethylene terephthalate (PET), a polyethylene and PVC laminate, a polyester and an aluminum and polyethylene laminate.

In accordance with a preferred embodiment of the present invention, the method further includes adding at least one of a food stabilizer and non-soluble fibers to the premix. Preferably, the food stabilizer includes lecithin. Preferably, the non-soluble fibers are selected from fructo-oligosaccharides (FOS), gluco-oligosaccharides (GOS), long-chain oligosaccharide polymers of fructose, long-chain oligosaccharide polymers of glucose and raffinose.

Preferably, the method further includes adding food additives selected from active ingredients, flavors and colors to the oil suspension. The active ingredients are preferably selected from minerals, vitamins, enzymes, amino acids, probiotic bacteria, fish oils, omega-3 fatty acids, fruits, algae extracts, vegetables, herbs and herb extracts, essential free fatty acids, joint active ingredients colostrum and carotenoids. The flavors are preferably selected from palatability enhancers.

In accordance with a preferred embodiment of the present invention, the food additives are added to the oil suspension in powdered form. In accordance with an alternative preferred embodiment of the present invention, the food additives are added to the oil suspension as an oil-based solution. Preferably, the molded food product is a solid at temperatures below 40° C.

In accordance with a preferred embodiment of the present invention, the method further includes sealing the packages with a cover sheet. Preferably, the cover sheet includes a material selected from laminated foil, polyethylene and polypropylene laminate or aluminum foil.

### DETAILED DESCRIPTION OF PREFERRED EMBODIMENTS

The present invention provides food products which are oil suspension-based, low in carbohydrates and low in water content. The food products of the present invention are suitable for human consumption and/or for animal consumption.

The food products of the present invention can be used, for example, as animal snacks, as high-protein and low carbohydrate sports nutrition snacks or as carriers for active ingredients, such as generic drugs, probiotic bacteria or vitamins. A more comprehensive list of active ingredients suitable for use with the present invention is provided below.

The food products of the present invention preferably contain 20% or less carbohydrates by weight, including sugar alcohols. More preferably, the food products of the present invention contain less than 10% carbohydrates by weight.

According to a preferred embodiment of the present invention, the food products of the present invention contain 1% or less water by weight. According to another preferred embodiment, the food products of the present invention contain 0.5% or less water by weight.

Preferably, the content of fat in the final food product is at least 10% by weight. More preferably, the content of fat in the final food product is between 25 and 45% by weight, most preferably between 30 and 35% by weight. For the purposes of the present specification and claims, the term “fat” includes within its scope animal and vegetable fats and oils.

Plant or animal fats suitable for use with the food product of the present invention include, for example, palm oil, cocoa butter, lard, coconut butter, beeswax, carnauba wax, tallow and any suitable combination thereof. Alternatively, any suitable plant or animal fat or combination of plant and/or animal fats may be used. It is appreciated that plant or animal fats generally include essentially no water or carbohydrates.

Preferably, when the food product is manufactured for human consumption, the fat is selected from plant fats such as palm oil, cocoa butter, coconut butter and combinations thereof. Alternatively, other plant or animal fats suitable for human consumption may be used.

Preferably, the content of protein in the final food product is between 30 and 80% by weight. More preferably, the content of protein in the final food product is between 30 and 70% by weight, most preferably between 50 and 65% by weight. For the purposes of the present specification and claims, the term “protein” includes within its scope animal and vegetable proteins.

Protein sources suitable for use with the food product of the present invention include, for example, poultry meal, meat meal, fish meal, dehydrated whey protein concentrate, soy protein concentrate, dried yeast, brewer's yeast, egg powder, animal digest and combinations thereof. Alternatively, other protein sources suitable for human or for animal consumption may be used.

Preferably, the food product of the present invention is prepared using any of the following protein sources, identified by their International Feed Name and International Feed Number (IFN), if available: primary dried yeast or dried yeast (IFN 7-05-533), brewer's dried yeast (IFN 7-05-527), egg product, poultry meal, poultry by-product meal (IFN 5-03-798), meat meal (IFN 5-00-385), fish meal (IFN 5-01-977), soy protein concentrate (IFN 5-32-183), dry whey protein concentrate (IFN 5-06-836), animal digest (IFN 5-06-935), or a combination thereof.

It is noted that an International Feed Number (IFN) is a number assigned to each International feed Name by the AAFCO (Association of American Food Control Officials) and can be used in order to find the composition of that feed name in the AAFCO databank. Tables including the composition of various feeds are found in “United States-Canadian Tables of Feed Composition: Nutritional Data for United States and Canadian Feeds, Third Revision, National Academy Press, 1982”, which is hereby incorporated by reference herein in its entirety.

Preferably, when the food product is manufactured for human consumption the protein source is selected from dehydrated whey protein concentrate, soy protein concentrate, brewer's yeast and egg powder. Alternatively, other protein sources suitable for human consumption may be used.

Preferably, when the food product is manufactured for animal consumption the protein source is selected from poultry meal, poultry by-product meal, fish meal, soy protein concentrate, brewer's yeast and egg powder. Alternatively, other protein sources suitable for animal consumption may be used.

Preferably, the protein source or combination of protein sources used for preparing the food product of the present invention has a water content of 1.5% or less and a carbohydrate content of 35% or less, by weight. More preferably, the protein source or combination of protein sources used for preparing the food product of the present invention has a water content of 1% or less and a carbohydrate content of 20% or less, by weight.

Preferably, if the water content of the protein source is higher than 1.5% by weight, the protein source is dried by conventional means.

The fat and the protein source together preferably constitute between 60 and 98% by weight of the final food product. More preferably, the fat and the protein source together constitute between 85 and 95% by weight of the final food product.

Preferably, the food product contains food additives, such as active ingredients, non-soluble fibers, food stabilizers, flavors and/or any other food additives. The food additives are preferably added in powdered form. Alternatively, some ingredients may be added as an oil-based solution.

Active ingredients suitable for use with the invention include, for example, minerals, water soluble vitamins such as vitamins B and C, oil soluble vitamins such as vitamins A, D and E, enzymes, amino acids, probiotic bacteria, fish oils, omega-3 fatty acids, fruits, algae extracts, vegetables, herbs and herb extracts, essential free fatty acids and joint active ingredients such as glucosamine sulfate, MSM (methyl-sulfonyl-methane), chondroitin, HA (hyaluronic acid), undenatured type-II collagen (such as UC-II®, available from Interhealth Neutraceuticals, Benicia, Calif., USA) calcium ascorbate (such as Ester C®, available from the Ester C Company, Bohemia, N.Y., USA), omega-6 fatty acids, hyaluronan and cetyl myristoleate.

Enzymes suitable for use as active ingredients include, but are not limited to pancreatic proteases, pancreatic lipases, pancreatic nucleases, pancreatic amylases, Trypsin, Chymotrypsin, Chymotrypsin B, Pancreatopeptidase, Carboxypeptidase A, Carboxypeptidase B, Glycerol ester hydrolase, Phospholipase A₂, Sterol ester hydrolase, Ribonuclease, Deoxyribonuclease, alpha-Amylase, Papain, Chymopapain, Bromelain, Ficin, beta-Amylase, Cellulase, beta-Galactosidase, Subtilopeptidase A, Betaine, Steapsin, Elastase, and Aspergillopeptidase A.

Probiotic bacteria suitable for use as active ingredients include, but are not limited to, lactobacilli, such as Lactobacillus acidophilus, Lactobacillus casei, Lactobacillus delbrueckii subsp bulgaricus (previously L. bulgaricus), Lactobacillus brevis, Lactobacillus cellobiosus, Lactobacillus curvatus, Lactobacillus fermentum, Lactobacillus lactis, Lactobacillus plantarum, Lactobacillus reuteri and Lactobacillus rhamnosus, streptococci, such as Streptococcus cremoris, Streptococcus salivarius subsp thermophilus (previously S. thermophilus), Enterococcus faecium (previously S. faecium), Streptococcus diacetylactis and Streptococcus intermedius, and bifidobacteria, such as Bifidobacterium bifidum, Bifidobacterium adolescentis, Bifidobacterium animalis, Bifidobacterium infantis, Bifidobacterium longum, Bifidobacterium thermophilum and Bifidobacterium lactis.

Preferably, when the food product is manufactured for animal consumption the active ingredients are selected from vitamins, probiotic bacteria, joint active ingredients, fish oil, omega-3 and omega-6 fatty acids, herbs and herb extracts, colostrum and carotenoids. Alternatively, other active ingredients suitable for animal consumption may be used.

Preferably, when the food product is manufactured for human consumption the active ingredients are selected from probiotic bacteria, oil soluble vitamins such as vitamins A, D and E, joint active ingredients and omega-3 and omega-6 fatty acids. Alternatively, other active ingredients suitable for human consumption may be used.

Non-soluble fibers suitable for use with the food product of the present invention include, but are not limited to fructo-oligosaccharides (FOS); gluco-oligosaccharides (GOS); other long-chain oligosaccharide polymers of fructose and/or glucose and the trisaccharide-raffinose.

Preferably, the non-soluble fibers suitable for use with the food product of the present invention include 5% or less water by weight.

Food additives suitable for use with the food product of the present invention, other than the active ingredients and the non-soluble fibers, include, but are not limited to food stabilizers such as lecithin, flavors including artificial sweeteners and palatability enhancers, and natural or artificial edible colors.

Preferably, the food additives suitable for use with the food product of the present invention, excluding non-soluble fibers, are added in a powder form or as an oil-based solution and therefore preferably have a water content of 3% or less by weight and a carbohydrate content of 5% or less by weight.

Preferably, any combination of fats, protein sources and food additives used to prepare the food product of the present invention has a water content of 1% or less and a carbohydrate content of 20% or less, by weight.

Preferably, the content of the food additives excluding the non-soluble fibers in the final food product is between 2 and 38% by weight. More preferably, the content of food additives excluding the non-soluble fibers in the final food product is between 2 and 20% by weight.

Preferably, the content of non-soluble fibers in the final food product is between 0 and 10% by weight.

According to a preferred embodiment of the present invention, the method of preparing the food products of the present invention comprises the following steps:

melting a plant or animal fat at a suitable temperature;

mixing the melted plant or animal fat with a protein source and optionally adding food stabilizers and/or non-soluble fibers to form a premix;

reducing the size of the particles in the premix to generate an oil suspension;

adding food additives such as food stabilizers, flavors, active ingredients and/or any additional food additives to the oil suspension;

molding the oil suspension by pouring it into cavities of packages; and

sealing the packages.

The plant or animal fat is preferably melted at a suitable temperature. For example, palm oil and lard are preferably melted at a temperature of between 42 and 45° C.; a mixture of coconut butter and beeswax is preferably melted at a temperature of between 48 and 54° C.; and cocoa butter and a mixture of tallow and palm oil are preferably melted at a temperature of between 40 and 45° C.

The step of reducing the size of the particles in the premix is preferably performed by wet grinding. Alternatively, the step of reducing the size of the particles in the premix is performed by another suitable method, such as dry grinding.

According to a preferred embodiment of the present invention the size of the particles in the premix is reduced to 100 microns or less. According to a more preferred embodiment, the size of the particles in the premix is reduced to 50 microns or less.

The packages wherein the oil suspension is molded are preferably packages produced by vacuum forming or punch forming of polyvinyl chloride (PVC), polyethylene terephthalate (PET), a polyethylene and PVC laminate, a polyester, an aluminum and polyethylene laminate, or any other suitable material.

The oil suspension is preferably a solid at temperatures below 40° C. Preferably, the oil suspension is not taken out of the package in which it is molded after solidifying and is marketed in the same package in which it is molded. Marketing in the same package in which the oil suspension is molded helps prevent decomposition of the active ingredients.

The packages are preferably sealed with laminated foil, polyethylene and polypropylene laminate or aluminum foil. Alternatively, the packages are sealed with any other suitable material. Methods of preparing and sealing the packages are described in U.S. Pat. No. 4,256,690, hereby incorporated by reference herein in its entirety. The present invention is also described with reference to the following non-limiting Examples:

### EXAMPLE 1

A Low Carbohydrate Oil Suspension-Based Animal Food Product

The recipe and the method of preparation are set out below:

Recipe

| Ingredient | % wt/wt |
|---|---|
| palm oil | 30 |
| poultry by-product meal | 65 |
| food additives | 5 |

Palm oil (EPS IMPEX Co., Sabah, Malaysia) is melted at 42° C. Poultry by-product meal (ECB Co., Treviglio, Italy) is added and mixed with the palm oil for 15 minutes. Particle size is reduced to less than 50 microns in dimension by wet grinding in a FrymaKoruma Stirrer Bead CoBall®-Mill, type MS 32, available from FrymaKoruma AG (Rheinfelden, Switzerland), to form an oil suspension. Milled food additives comprising C'SENS 9P palatability enhancer (SPF, Elven, France) and vitamin E (0.1% w/w) are mixed in. The oil suspension is poured into cavities of packages produced by vacuum forming of PVC at a thickness 400 microns and the packages are heat sealed with laminated foil.

### EXAMPLE 2

A Low Carbohydrate Oil Suspension-Based Animal Food Product

The recipe and the method of preparation are set out below:

Recipe

| Ingredient | % wt/wt |
|---|---|
| lard | 30 |
| fish meal | 65 |
| food additives | 5 |

Lard (Otto Schachinger GmbH, Enzersdorf, Austria) is melted at 42° C. Fish meal (ECB Co., Treviglio, Italy) is added and premixed with the melted lard for 15 minutes. Particle size is reduced to less than 30 microns in dimension by wet grinding in a Netzsch Type KE 50 ball mill, available from NETZSCH Werbe- and Service-GmbH (Selb, Germany), to form an oil suspension. Milled food additives comprising C'SENS 9P palatability enhancer (SPF, Elven, France) and vitamin E (0.1% w/w) are mixed in. The oil suspension is poured into cavities of packages produced by vacuum forming of PET at a thickness 250 microns and the packages are heat sealed with laminated foil.

### EXAMPLE 3

A Low Carbohydrate Oil Suspension-Based Animal Food Product

The recipe and the method of preparation are set out below:

Recipe

| Ingredient | % wt/wt |
|---|---|
| coconut butter | 27 |
| beeswax | 3 |
| dehydrated whey protein concentrate | 65 |
| food additives | 5 |

Coconut butter (Artisana, Berkeley, Calif., USA) is melted with beeswax (Koster Keunen, Bladel, The Netherlands) at 50° C. Dehydrated whey protein concentrate (BA'EMEK, Afula, Israel) is added to the melted oil suspension. The ingredients are premixed and particle size is reduced to less than 50 microns in a FrymaKoruma Stirrer Bead CoBall®-Mill, type MS 32, available from FrymaKoruma AG (Rheinfelden, Switzerland), to form an oil suspension. The oil suspension is pumped into a heated jacket reactor available from Mazden (Buenos Aires, Argentina), milled food additives comprising C'SENS 9P palatability enhancer (SPF, Elven, France) and vitamin E (0.1% w/w) are added and the ingredients are mixed in a Turbo Blender IKA-ROTOTRON, available from IKA-Werke GmbH & Co. KG (Staufen, Germany). The suspension is poured into cavities of packages produced by vacuum forming of 20 micron polyethylene and 120 micron PVC laminate. The packages are heat sealed with a 10 micron polyethylene and 50 micron polypropylene laminate.

### EXAMPLE 4

A Low Carbohydrate Oil Suspension-Based Human Food Product

The recipe and the method of preparation are set out below:

Recipe

| Ingredient | % wt/wt |
|---|---|
| tallow | 22 |
| palm oil | 22 |
| soy protein concentrate | 50 |
| food additives | 6 |

Tallow (Chemical Associates, Copley, Ohio, USA) is melted together with palm oil (EPS IMPEX Co., Sabah, Malaysia) at 42° C. Soy protein concentrate (USSEC, St. Louis, Mo., USA) is added to the melted oil suspension. The ingredients are premixed and particle size is reduced to less than 40 microns in a Wiener W-85 Ball mill, available from Duyvis Wiener B.V. (Zaanstad, the Netherlands), to form a suspension. The suspension is pumped into an Olimpic SR scraped surface heat exchanger available from Rossi & Catelli (Parma, Italy), or into a Contherm scraped-surface heat exchanger model: 6×6 available from Alfa Laval (Lund, Sweden), preferably heated to 85-90° C. for at least 5 minutes. The heated suspension is cooled down to 40° C. in the scraped surface heat exchanger. The cooled suspension is pumped to a heated jacket reactor available from Mazden (Buenos Aires, Argentina) and milled food additives comprising C'SENS 9P palatability enhancer (SPF, Elven, France) and vitamin E (0.1% w/w) are added and mixed in a Turbo Blender IKA-ROTOTRON, available from IKA-Werke GmbH & Co. KG (Staufen, Germany). The suspension is poured into cavities of packages produced by punch forming of 150 micron polyester, 20 micron aluminum and 20 micron polyethylene laminate. The packages are heat sealed with a 20 micron aluminum foil laminate.

### EXAMPLE 5

A Low Carbohydrate Oil Suspension-Based Human Food Product Including Non-Soluble Fibers

The recipe and the method of preparation are set out below:

Recipe

| Ingredient | % wt/wt |
|---|---|
| cocoa butter | 33 |
| whey protein concentrate | 55 |
| polyfructose | 10 |
| other food additives | 2 |

Cocoa butter (Amrut International, Gujarat, India) is melted at 42° C. A whey protein concentrate which has especially low carbohydrate content, such as K-PRO 150 available from BA'EMEK (Afula, Israel), is used in this example. If the water content of the whey protein concentrate is higher than 1% by weight, the whey protein concentrate is dried by conventional means until the water content thereof is below 0.5% by weight. The dry whey protein concentrate and polyfructose (Roton International Corp., Jiangxi, China) are added to the melted cocoa butter. The ingredients are premixed and particle size is reduced to less than 50 microns in dimension in a Wiener W-85 Ball mill, available from Duyvis Wiener B.V. (Zaanstad, the Netherlands), to form a suspension. The suspension is pumped into a heated jacket reactor available from Mazden (Buenos Aires, Argentina), and milled food additives comprising C'SENS 9P Palatability enhancer (SPF, Elven, France) are added and mixed in a Turbo Blender IKA-ROTOTRON, available from IKA-Werke GmbH & Co. KG (Staufen, Germany). The suspension is cooled down to 32° C. and then heated to 38° C. The heated suspension is poured into cavities of packages produced by vacuum forming of 20 micron polyethylene and 120 micron PVC laminate. The packages are heat sealed with a 10 micron polyethylene and 50 micron polypropylene laminate. The sealed packages are cooled in a cooling tunnel to 12° C. and then allowed to return to room temperature.

### EXAMPLE 6

A Low Carbohydrate Oil Suspension-Based Animal Multivitamin Supplement

The recipe and the method of preparation are set out below:

Recipe

| Ingredient | % wt/wt |
|---|---|
| palm oil | 26 |
| poultry by-product meal | 45.9 |
| egg powder | 6 |
| yeast extract | 5 |
| lecithin | 1 |
| carnauba wax | 6 |
| multivitamin blend | 7 |
| other food additives | 3.1 |

Palm oil (EPS IMPEX Co., Sabah, Malaysia) and lecithin (American Lecithin Company, Oxford, Conn., USA) are melted at 65° C. Protein poultry meal (ECB, Co., Treviglio, Italy), egg powder (Cham Food, Ltd., Hadera, Israel), yeast extract (Ohly GmbH, Hamburg, Germany) and carnauba wax (Koster Keunen, Bladel, The Netherlands) are added and mixed with the palm oil and lecithin for 15 minutes. Particle size is reduced to less than 100 microns in dimension by wet grinding in a FrymaKoruma Toothed Colloid Mill, type MZ 130, available from FrymaKoruma AG (Rheinfelden, Switzerland), to form an oil suspension. Milled multivitamin blend and food additives comprising C'SENS 9P palatability enhancer (SPF, Elven, France) are mixed in. The oil suspension is poured into cavities of packages produced by vacuum forming of PVC at a thickness 400 microns and the packages are heat sealed with laminated foil.

### EXAMPLE 7

A Low Carbohydrate Oil Suspension-Based Animal Joint Care Supplement

The recipe and the method of preparation are set out below:

Recipe

| Ingredient | % wt/wt |
|---|---|
| palm oil | 27 |
| poultry by-product meal | 23.9 |
| egg powder | 6 |
| yeast extract | 5 |
| lecithin | 1 |
| carnauba wax | 2 |
| glucosamine-HCl | 20 |
| methyl sulfonylmethane | 10 |
| chondroitin | 5 |
| other food additives | 0.1 |

Palm oil (EPS IMPEX Co., Sabah, Malaysia) and lecithin (American Lecithin Company, Oxford, Conn., USA) are melted at 65° C. Protein poultry meal (ECB Co., Treviglio, Italy), egg powder (Cham Food, Ltd., Hadera, Israel), yeast extract (Ohly GmbH, Hamburg, Germany) and carnauba wax (Koster Keunen, Bladel, The Netherlands) are added and premixed with the palm oil and Lecithin for 15 minutes. Particle size is reduced to less than 100 microns in dimension by wet grinding in a FrymaKoruma Toothed Colloid Mill, type MZ 130, available from FrymaKoruma AG (Rheinfelden, Switzerland), to form an oil suspension. Milled glucosamine (Golden Shell Biochemical, Yuhuan, China), methyl sulfonylmethane (Panjin Far East Jinxing Chemical Co., Panjin, China), chondroitin (Nutristar Inc. Ft. Lee, N.J., USA) and food additives comprising C'SENS 9P palatability enhancer (SPF, Elven, France) are mixed in. The oil suspension is poured into cavities of packages produced by vacuum forming of PVC at a thickness 400 microns and the packages are heat sealed with laminated foil.

### EXAMPLE 8

A Low Carbohydrate Oil Suspension-Based Animal Essential Fatty Acid Supplement

The recipe and the method of preparation are set out below:

Recipe

| Ingredient | % wt/wt |
|---|---|
| palm oil | 25 |
| poultry by-product meal | 43 |
| egg powder | 6 |
| yeast extract | 5 |
| lecithin | 1 |
| carnauba wax | 6 |
| beeswax | 4 |
| fish oil | 6.6 |
| other food additives | 3.4 |

Palm oil (EPS IMPEX Co., Sabah, Malaysia) and lecithin (American Lecithin Company, Oxford, Conn., USA) are melted at 65° C. Protein poultry meal (ECB Co., Treviglio, Italy), egg powder (Cham Food, Ltd., Hadera, Israel), yeast extract (Ohly GmbH, Hamburg, Germany), beeswax (Koster Keunen, Bladel, The Netherlands) and carnauba wax (Koster Keunen, Bladel, The Netherlands) are added and premixed with the palm oil and lecithin for 15 minutes. Particle size is reduced to less than 100 microns in dimension by wet grinding in a FrymaKoruma Toothed Colloid Mill, type MZ 130, available from FrymaKoruma AG (Rheinfelden, Switzerland), to form an oil suspension. Fish oil (Denomega, Sarpsborg, Norway) and food additives comprising C'SENS 9P palatability enhancer (SPF, Elven, France) are mixed in. The oil suspension is poured into cavities of packages produced by vacuum forming of PVC at a thickness 400 microns and the packages are heat sealed with laminated foil.

### EXAMPLE 9

A Low Carbohydrate Oil Suspension-Based Animal Prebiotic and Probiotic Supplement

The recipe and the method of preparation are set out below:

Recipe

| Ingredient | % wt/wt |
|---|---|
| palm oil | 27 |
| poultry by-product meal | 43.9 |
| egg powder | 6 |
| yeast extract | 5 |
| lecithin | 1 |
| beeswax | 3 |
| CaCO₃ | 4 |
| fructo oligo saccharides (FOS) | 6 |
| freeze dried bacteria | 1 |
| other food additives | 3.1 |

Palm oil (EPS IMPEX Co., Sabah, Malaysia) and lecithin (American Lecithin Company, Oxford, Conn., USA) are melted at 65° C. Protein poultry meal (ECB Co., Treviglio, Italy), Egg powder (Cham Food, Ltd., Hadera, Israel), Yeast extract (Ohly GmbH, Hamburg, Germany) and beeswax are added and premixed with the palm oil and Lecithin for 15 minutes. Particle size is reduced to less than 100 microns in dimension by wet grinding in a FrymaKoruma Toothed Colloid Mill, type MZ 130, available from FrymaKorurna AG (Rheinfelden, Switzerland), to form an oil suspension. Freeze dried bacteria (Danisco, Copenhagen, Denmark), calcium carbonate, FOS (Victory Biology Engineering Co., Shenzhen, China) and food additives comprising C'SENS 9P palatability enhancer (SPF, Elven, France) are mixed in. The oil suspension is poured into cavities of packages produced by vacuum forming of PVC at a thickness 400 microns and the packages are heat sealed with laminated foil.

### EXAMPLE 10

A Low Carbohydrate Oil Suspension-Based Animal Immune Support Supplement

The recipe and the method of preparation are set out below:

Recipe

| Ingredient | % wt/wt |
|---|---|
| palm oil | 26 |
| poultry by-product meal | 51.5 |
| egg powder | 6 |
| yeast extract | 5 |
| lecithin | 1 |
| carnauba wax | 5 |
| pure colostrum | 2 |
| other food additives | 3.5 |

Palm oil (EPS IMPEX Co., Sabah, Malaysia) and lecithin (American Lecithin Company, Oxford, Conn., USA) are melted at 65° C. Protein poultry meal (ECB Co., Treviglio, Italy), egg powder (Cham Food, Ltd., Hadera, Israel), yeast extract (Ohly GmbH, Hamburg, Germany) and carnauba wax (Koster Keunen, Bladel, The Netherlands) are added and premixed with the palm oil and lecithin for 15 minutes. Particle size is reduced to less than 100 microns in dimension by wet grinding in a FrymaKoruma Toothed Colloid Mill, type MZ 130, available from FrymaKoruma AG (Rheinfelden, Switzerland). Colostrum (Synertek, Cheyenne, Wyo., USA) and food additives comprising C'SENS 9P palatability enhancer (SPF, Elven, France) are mixed in. The oil suspension is poured into cavities of packages produced by vacuum forming of PVC at a thickness 400 microns and the packages are heat sealed with laminated foil.

### EXAMPLE 11

A Low Carbohydrate Oil Suspension-Based Animal Pacifying Supplement

The recipe and the method of preparation are set out below:

Recipe

| Ingredient | % wt/wt |
|---|---|
| palm oil | 26 |
| poultry by-product meal | 43.5 |
| egg powder | 6 |
| yeast extract | 5 |
| lecithin | 1 |
| carnauba wax | 5 |
| valerian | 5 |
| herb extracts | 5 |
| other food additives | 3.5 |

Palm oil (EPS IMPEX Co., Sabah, Malaysia) and lecithin (American Lecithin Company; Oxford, Conn., USA) are melted at 65° C. Protein poultry meal (ECB Co., Treviglio, Italy), egg powder (Cham Food, Ltd., Hadera, Israel), yeast extract (Ohly GmbH, Hamburg, Germany) and carnauba wax (Koster Keunen, Bladel, The Netherlands) are added and premixed with the palm oil and lecithin for 15 minutes. Particle size is reduced to less than 100 microns in dimension by wet grinding in a FrymaKoruma Toothed Colloid Mill, type MZ 130, available from FrymaKoruma AG (Rheinfelden, Switzerland). Valerian (Pharmonta Dr. Fischer GmbH, Gratwein, Austria), herb extracts comprising chamomile, St. Johns wort, hops and ginger (Anlit Extracts, Granot, Israel) and food additives comprising C'SENS 9P palatability enhancer (SPF, Elven, France) are mixed in. The oil suspension is poured into cavities of packages produced by vacuum forming of PVC at a thickness 400 microns and the packages are heat sealed with laminated foil.

### EXAMPLE 12

### A low carbohydrate oil suspension-based animal eye & vision support supplement. The recipe and the method of preparation are set out below:

Recipe

| Ingredient | % wt/wt |
|---|---|
| palm oil | 26 |
| poultry by-product meal | 48.3 |
| egg powder | 6 |
| yeast extract | 5 |
| lecithin | 1 |
| carnauba wax | 5 |
| carotenoids | 0.2 |
| herb extracts | 5 |
| other food additives | 3.5 |

Palm oil (EPS IMPEX Co., Sabah, Malaysia) and Lecithin are melted at 65° C. Protein poultry meal (ECB Co., Treviglio, Italy), Egg powder (Cham Food, Ltd., Hadera, Israel), Yeast extract (Ohly GmbH, Hamburg, Germany) and Carnauba wax (Koster Keunen, Bladel, The Netherlands) are added and premixed with the palm oil and Lecithin for 15 minutes. Particle size is reduced to less than 100 microns in dimension by wet grinding in a FrymaKoruma Toothed Colloid Mill, type MZ 130, available from FrymaKoruma AG (Rheinfelden, Switzerland). Carotenoids, (Apin Chemicals, Abingdon, UK), herb extracts comprising chamomile, St. Johns wort, hops and ginger (Anlit Extracts, Granot, Israel) and food additives comprising C'SENS 9P palatability enhancer (SPF, Elven, France) are mixed in. The oil suspension is poured into cavities of packages produced by vacuum forming of PVC at a thickness 400 microns and the packages are heat sealed with laminated foil.

It will be appreciated by persons skilled in the art that the present invention is not limited by what has been particularly shown and described hereinabove. Rather the scope of the present invention includes both combinations and sub-combinations of various features of the invention and modifications thereof which may occur to persons skilled in the art upon reading the foregoing description and which are not in the prior art.

1. A molded food product comprising:
25-45% fat by weight;
30-70% protein by weight; and
not more than 20% carbohydrate by weight.

2. The molded food product of claim 1, wherein the water content of said product is less than 1% by weight.

3. (canceled)

4. The molded food product of claim 1, which comprises not more than 10% carbohydrate by weight.

5. The molded food product of claim 1, which comprises 30-35% fat by weight.

6. The molded food product of claim 1, wherein said fat is selected from palm oil, cocoa butter, lard, coconut butter, beeswax, carnauba wax, tallow and combinations thereof.

7. (canceled)

8. The molded food product of claim 1, which comprises 50-65% protein by weight.

9. The molded food product of claim 1, wherein said protein is selected from poultry meal, poultry by-product meal, meat meal, fish meal, dehydrated whey protein concentrate, soy protein concentrate, dried yeast, brewer's yeast, egg powder, animal digest and combinations thereof.

10. (canceled)

11. (canceled)

12. The molded food product of claim 1, wherein the weight percent in said product of said fat and said protein combined is from 60-98%.

13. (canceled)

14. The molded food product of claim 1, further comprising food additives selected from active ingredients, non-soluble fibers, food stabilizers, flavors and colors.

15. The molded food product of claim 14, wherein said product comprises 2-38% by weight food additives, excluding said non-soluble fibers.

16. (canceled)

17. The molded food product of claim 14, wherein said active ingredients are selected from minerals, vitamins, enzymes, amino acids, probiotic bacteria, fish oils, omega-3 fatty acids, fruits, algae extracts, vegetables, herbs and herb extracts, essential free fatty acids, glucosamine, chondroitin, methylsulfonyl methane, undenatured type-II collagen, colostrum and carotenoids.

18. (canceled)

19. (canceled)

20. The molded food product of claim 14, wherein said non-soluble fibers are selected from fructo-oligosaccharides (FOS), gluco-oligosaccharides (GOS), long-chain oligosaccharide polymers of fructose, long-chain oligosaccharide polymers of glucose and raffinose.

21. The molded food product of claim 14, wherein said product comprises not more than 10% non-soluble fibers by weight.

22. (canceled)

23. (canceled)

24. The molded food product of claim 1, which is in the form of an oil suspension.

25. The molded food product of claim 1, wherein the particles in said product have a maximum dimension of not more than 100 microns.

26. (canceled)

27. The molded food product of claim 1, which is a solid at temperatures below 40° C.

28-30. (canceled)

31. A method of forming a molded food product comprising:
melting a plant or animal fat;
mixing said melted plant or animal fat with a protein source to form a premix;
reducing the size of the particles in said premix to generate an oil suspension; and
pouring the oil suspension into cavities of packages to form said molded food product.

32. The method of claim 31, wherein said plant or animal fat is selected from palm oil, cocoa butter, lard, coconut butter, beeswax, carnauba wax, tallow and combinations thereof.

33. The method of claim 31, wherein said protein source is selected from poultry meal, poultry by-product meal, meat meal, fish meal, dehydrated whey protein concentrate, soy protein concentrate, dried yeast, brewer's yeast, egg powder, animal digest and combinations thereof.

34. The method of claim 31, wherein said reducing is performed by wet grinding.

35. The method of claim 31, wherein the size of said particles is reduced to not more than 100 microns.

36. (canceled)

37. (canceled)

38. The method of claim 31, further comprising adding at least one of a food stabilizer and non-soluble fibers to said premix.

39. (canceled)

40. The method of claim 38, wherein said non-soluble fibers are selected from fructo-oligosaccharides (FOS), gluco-oligosaccharides (GOS), long-chain oligosaccharide polymers of fructose, long-chain oligosaccharide polymers of glucose and raffinose.

41. The method of claim 31, further comprising adding food additives selected from active ingredients, flavors and colors to said oil suspension.

42. The method of claim 41, wherein said active ingredients are selected from minerals, vitamins, enzymes, amino acids, probiotic bacteria, fish oils, omega-3 fatty acids, fruits, algae extracts, vegetables, herbs and herb extracts, essential free fatty acids, glucosamine, chondroitin, methylsulfonyl methane, undenatured type-II collagen, colostrum and carotenoids.

43. (canceled)

44. The method of claim 41, wherein said food additives are added to said oil suspension in powdered form.

45. The method of claim 41, wherein said food additives are added to said oil suspension as an oil-based solution.

46. The method of claim 31, wherein said molded food product is a solid at temperatures below 40° C.

47. (canceled)

48. (canceled)
